(19) United States
(12) Patent Application Publication
(10) Pub. No.: US 2006/0212020 A1
(43) Pub. Date: Sep. 21, 2006

(54) Sample collection system with caspase inhibitor

(76) Inventors: Lynne Rainen, Maplewood, NJ (US); Andrca Liebmann-Vinson, Wake Forest, NC (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 10/530,824

(22) PCT Filed: Oct. 10, 2003
(86) PCT No.: PCT/US03/32261

§ 371 Date: Oct. 19, 2005

(30) Foreign Application Priority Data
Oct. 10, 2002 (US) 60417531

(51) Int. Cl. A61B 19/00

(52) U.S. Cl. 604/403

(57) ABSTRACT

A collection container and a method for collecting a biological sample, particularly whole blood, includes at least one stabilizing agent in an amount effective to inhibit apoptosis. The stabilizing agent comprises or consists of one or more caspase inhibitors.

### FIELD OF THE INVENTION

The present invention is directed to a method and device for collecting and stabilizing a biological sample, directly from a patient and/or cultured cells, either from animals or humans.

### BACKGROUND OF THE INVENTION

In clinical diagnostic and clinical research settings, it has often been necessary to collect biological samples such a whole blood, red blood cell concentrates, platelet concentrates, leukocyte concentrates, tissue, bone marrow apirates, cerebral spinal fluid, feces, urine, saliva, oral secretions, nasal secretions and the like in various containers or tubes for subsequent analysis or in vitro culture. Typically, the samples must then be transported to a different location, such as a laboratory, where personnel conduct specific tests on or manipulate the samples for future testing or implantation.

Generally, a considerable amount of time elapses between obtaining the sample and analyzing or manipulating it. A common and recurring problem, therefore, is the maintenance of the biological sample in a manner that prevents degradation, alteration or destruction of essential materials during the manipulations and/or preparations preceding analysis or implantation of the biological sample as a test specimen.

### SUMMARY OF THE INVENTION

All cells and tissues undergo a process of apoptosis, or programmed cell death (Kerr, Wyllie, and Currie, 1972 Brit J. Cancer 26:239) as a natural process in development and morphogenesis to remove unwanted or aged cells. Cells undergoing apoptosis are morphologically and biochemically distinguishable from cells involved in necrosis, which is associated with acute injury to cells. Apoptosis is characterized by nuclear chromain condensation, DNA fragmentation, cell membrane alterations, caspase activity, cytoplasmic shrinking, dilated endoplasmic reticulum, and membrane blebbing.

Apoptotic death can be triggered by a wide variety of stimuli, and not all cells necessarily will die in response to the same stimulus. Among the more studied death stimuli is DNA damage by irradiation or drugs used for cancer chemotherapy. Some hormones such as corticosteroids lead to death in particular cells (e.g., thymocytes), although other cell types may be stimulated. Apoptosis is also evident in blood samples collected by phlebotomy and tissue samples.

Biochemical correlates of these morphological features have emerged during the subsequent years of study of this phenomenon. The first and most dramatic is DNA fragmentation, which was described by Wyllie in 1980. When DNA from apoptotically dying cells was subjected to agarose gel electrophoresis, ladders with ˜200 bp repeats were observed, corresponding histone protection in the nucleosomes of native chromatin. Subsequent pulsed field gel techniques have revealed earlier DNA cleavage patterns into larger fragments. Since even a few double stranded DNA breaks will render the cell unable to undergo mitosis successfully, such DNA fragmentation can be regarded as a biochemical definition of death.

Changes in the cell membrane in the apoptotic cell trigger phagocytosis by non-activated macrophages. Apoptotic cells lose the normal phospholipid asymmetry in their plasma membrane, as manifested by the exposure of normally inward-facing phosphatidyl serine on the external face of the bilayer. Macrophages can recognize this exposed lipid headgroup via an unknown receptor, triggering phagocytosis and elimination of these damaged cells from the organism.

Apoptosis in granulocytes present in whole blood samples post phlebotomy is manifested by loss of key features of this important cell type. CD16b (a granulocyte cell surface marker), oxidative burst function, membrane lipid polarity and hypodiploidy all decline, degrading the quality of the sample for analysis. Consequently, aberrant or inaccurate analytical test results which depend on these cell paramaters can occur with whole blood samples analyzed after 8 hours from the time of phlebotomy.

Another biochemical hallmark of apoptotic death is the activation of caspases, which are cysteine proteases related to ced-3, the “death gene” of the nematode Caenorhabditis elegans. Caspases seem to be widely expressed in an inactive proenzyme form in most cells. Their proteolytic activity is characterized by their unusual ability to cleave proteins at aspartic acid residues, although different caspases have different fine specificities involving recognition of neighboring amino acids. Active caspases can often activate other pro-caspases, allowing initiation of a protease cascade. Persuasive evidence that these proteases are involved in most examples of apoptotic cell death has come from the ability of specific caspase inhibitors to block cell death, as well as the demonstration that knockout mice lacking caspase 3, 8 and 9 fail to complete normal embryonic development.

In the area of blood collection, a common additive generally used in blood samples prior to centrifuging to separate the blood into cell layers is an anticoagulation additive. Typically, the anticoagulation additive is a potassium or sodium salt of ethylene diamine tetraacetic acid (EDTA), a buffered citrate, or heparin in an aqueous solution or crystalline coating of the interior of the collection vessel. Blood collection tubes containing an anticoagulant are commercially manufactured and sold. An example of such a tube is disclosed in U.S. Pat. No. 5,667,963 to Smith et al. These additives do not specifically halt apoptosis nor do anything to protect cell morphology or function.

The present invention, therefore, is directed to methods and devices for collecting a biological sample for the purpose of preserving cell morphology, viability and function. More particularly, the invention is directed to collection containers and to a method of collecting a biological sample and immediately contacting the sample with a stabilizing additive or combination of additives to inhibit endogenous caspases for the subsequent analysis or in vitro culture of the cells in the biological sample. The stabilizing agent or agents of the invention is a suitable mixture that is able to inhibit, prevent or reduce the occurrence of apoptosis and apoptotic events during storage or culture of the biological sample.

Accordingly, a primary aspect of the present invention is to provide a method and device for collecting a biological sample, directly from a patient or animal in the presence of a stabilizer or stabilizer mixture capable of inhibiting endogenous caspases for analysis of morphology or function of whole cells, cell constituents, or tissue, or maintaining the viability of same for in vitro culture of cells or tissue. The stabilizing additive is present in an effective amount to stabilize the biological sample and to inhibit endogenous caspases for analysis of cells, cell constituents, tissue or culture. Desirably, the sample is whole blood or a tissue sample.

One aspect of the present invention is to prepare a biological sample that is stable at room temperature for extended periods of time with little or no degradation in cell morphology, function, or composition. Accordingly, a method is provided for producing a biological sample that is stable at room temperature for extended periods of time with little or no incidence of morphological changes, cell membrane degradation, DNA fragmentation, or loss of cell function or viability.

A further aspect of the invention is to provide a method and device for inhibiting or eliminating incidence of morphological changes, cell membrane degradation, DNA fragmentation, or loss of cell function or viability.

Another aspect of the invention is to provide a collection container for receiving and collecting a biological sample where the container is pre-charged with a measured quantity of a stabilizing agent or mixture of agents. The stabilizing agent may be supplied in the form of a liquid, a liquid or solid aerosol, a pellet, a powder or a gel to any surface of the container.

A further aspect of the present invention is to provide a method for stabilizing a biological sample, particularly whole blood or a component thereof, immediately upon collection from the patient to inhibit or prevent sample degradation when the sample is stored at various temperatures.

Another aspect of the present invention is to provide an evacuated container that is supplied with an effective amount of a stabilizing agent, where the container has an internal pressure sufficiently low to draw a predetermined volume of a biological sample into the container.

Still another aspect of the present invention is to provide a blood collection container for collecting an amount of blood and mixing the blood with a stabilizing agent or mixture of agents at the point of collection to produce a blood sample that is stable by preventing degradation of the sample such that analysis of cell morphology, function or in vitro culture of the sample can be conducted at a later time.

The aspects of the invention are attained by providing an apparatus for collecting a biological sample. The apparatus generally includes a container comprising at least one interior wall that defines a reservoir portion for containing a volume of a biological sample and at least one opening in communication with the reservoir portion. The container includes at least one stabilizing agent in an effective amount to preserve the biological sample and prevent or inhibit or eliminate incidence of morphological changes, cell membrane degradation, DNA fragmentation, or loss of cell function or viability. Preferably, the container is pre-treated with the stabilizing agent prior to collection of the sample.

The aspects of the invention are further attained by providing a method of preparing a stable biological sample comprising providing a sample collection container. Desirably, the container has a side wall and a bottom defining an internal chamber where the interior of the container contains at least one stabilizing agent in an amount sufficient to prevent or eliminate incidence of morphological changes, cell membrane degradation, DNA fragmentation, or loss of cell function or viability. The container may have attributes associated with promotion of in vitro cell culture. A biological sample is obtained and immediately introduced into the container, and the biological sample is mixed with the stabilizing agent to form a stabilized biological sample.

The aspects of the invention are also attained by providing a method of collecting and stabilizing a whole blood sample. The method comprises providing a sample collection container having a side wall and a bottom forming an internal chamber. The container is provided with an effective amount of a stabilizing agent to stabilize cells in the whole blood sample. The internal chamber has pressure less than atmospheric pressure. A whole blood sample is collected directly from a patient in the collection container, and the blood sample is mixed with the stabilizing agent to form a stable whole blood sample. As the biological sample is drawn into the collection device, it is immediately exposed to the stabilizing agent, and the process of protecting cell morphology, membrane integrity and function would begin immediately upon introduction of the sample.

The aspects of the invention are also attained by providing a method of collecting and stabilizing a tissue or bone marrow or body fluid aspirate sample. The method comprises providing a sample collection container having a side wall and a bottom forming an internal chamber. The container is provided with an effective amount of a stabilizing agent or mixture of agents to stabilize cells in tissue or aspirate sample. A tissue or bone marrow or body fluid aspirate sample is collected directly from a patient in the collection container, and the sample is mixed with the stabilizing agent to form a stable biological sample. As the biological sample is introduced into the collection device, it is immediately exposed to the stabilizing agent, and the process of protecting cell morphology, membrane integrity and function would begin immediately upon introduction of the sample. The container may also be provided with attributes that promote the culture or growth of cells. These attributes may include but not be limited to surface charge of interior surfaces of the container, porous membranes, cell nutrient media, or artificial scaffolding structures.

The method and collection device of the present invention have several distinct advantages. One advantage of the collection device is the offering of a system, preferably a closed system, that includes the stabilizing agent and which protects the sample from deleterious exposures. Still another advantage is routine line production of such collection devices, whereby quality control measures and procedures are applied to the product. Yet another advantage is the standardization of such collection devices where no industry standards currently exist. Moreover, the relevance of cell or tissue research and analysis is increased by preserving and being able to characterize and study cells in a state that is as close to the in vivo state as possible.

These aspects, advantages and other salient features of the present invention will become more apparent from the following detailed description of the invention, particularly when considered in conjunction with the drawings.

### BRIEF DESCRIPTION OF THE DRAWINGS

FIG. 1 is a perspective view of a typical blood collection tube.

FIG. 2 is a perspective view of a tissue culture vessel.

FIGS. 3A and 3B show a sample collection assembly.

FIG. 4 is a longitudinal sectional view of a syringe.

FIG. 5 is a longitudinal sectional view of another embodiment of a syringe.

FIG. 6 is a perspective view illustrating a blood collecting bag.

### DETAILED DESCRIPTION OF THE INVENTION

While this invention is satisfied by embodiments in many different forms, there will herein be described in detail preferred embodiments of the invention, with the understanding that the present disclosure is to be considered as exemplary of the principles of the invention and is not intended to limit the invention to the embodiments illustrated and described. Numerous variations may be made by persons skilled in the art without departure from the spirit of the invention. The scope of the invention will be measured by the appended claims and their equivalents.

The present invention is directed to a method and device for stabilizing a biological sample to better enable analysis and in vitro culture of cells and tissues. More particularly, the present invention is directed to a method and device for inhibiting apoptosis in a biological sample during storage or culture. According to the present invention, the device comprises a container containing an amount of a stabilizing agent for admixing with a biological sample immediately on collection of the sample. Also according to the present invention, the method comprises providing a sample collection container containing a stabilizing agent in an amount sufficient to inhibit apoptosis and adding to the container a biological sample.

The biological sample is any body fluid or tissue sample withdrawn from a patient. Typically, the biological sample is whole blood or a component thereof, including umbilical cord blood or placental blood. Examples of other biological samples include cell-containing compositions such as red blood cell concentrates, platelet concentrates, leukocyte concentrates, urine, bone marrow apirates, cerebral spinal fluid, tissue, fine needle organ or lesion aspirates, feces, saliva and oral secretions, nasal secretions lymphatic fluid and the like.

The sample collection system of the present invention can encompass any collection device including, but not limited to, tubes such as test tubes and centrifuge tubes; closed system blood collection devices, such as collection bags; syringes, especially pre-filled syringes; catheters; microtiter and other multi-well plates; arrays; tubing; laboratory vessels such as flasks, spinner flask, roller bottles, vials, microscope slides, microscope slide assemblies, coverslips, films and porous substrates and assemblies; pipettes and pipette tips, etc.; and other containers suitable for holding a biological sample. The interior of the vessel may be treated with the stabilizing agent.

Plastic or glass is often used to manufacture the collection device used in the present invention. Some preferred materials used to manufacture the collection device include polypropylene, polyethylene, polyethyleneterephthalate, polystyrene, polycarbonate and cellulosics. More expensive plastics such as polytetrafluoroethylene and other fluorinated polymers may also be used. In addition to the materials mentioned above, examples of other suitable materials for the collection devices used in the present invention include polyolefins, polyamides, polyesters, silicones, polyurethanes, epoxies, acrylics, polyacrylates, polysulfones, polymethacrylates, PEEK, polyimide and fluoropolymers such as PTFE Teflon®, FEP Teflon®, Tefzel®, poly(vinylidene fluoride), PVDF and perfluoroalkoxy resins. Glass products including silica glass are also used to manufacture the collection devices. One exemplary glass product is PYREX® (available from Corning Glass, Corning, N.Y.). Ceramic collection devices can be used according to embodiments of the invention. Cellulosic products such as paper and reinforced paper containers can also be used to form collection devices according to the invention.

The stabilizing agent of the invention is a suitable agent that is able to inhibit caspase activity and the resultant apoptotic events during storage of a biological sample. The agent stabilizes the biological sample, such as a blood sample, to produce a stable composition that inhibits or prevents apoptosis present in the biological sample. In accordance with the present invention, the collection device is pre-treated with the stabilizing agent, preferably by the manufacturer, and is packaged in a ready-to-use form. Typically, the packaged collection device is sterile and is also packaged in sterile packaging materials.

The present invention could be used by clinical laboratories, pharmaceutical companies, biotechnology companies, contract research organizations, university researchers, research hospitals and any institution and individual who is interested in studying or analyzing cells or tissues. The present invention would enable researchers and laboratorians to conveniently and readily protect cellular samples for downstream analysis. The collection device according to the present invention would serve as a front-end sample collection device aiding analytical and processing objectives including, both to add not limited to the following: flow cytometry, multiplexed bead assays cell surface marker identification and analysis, routine hematology assays including CBC and white blood cell differential, HLA typing, cord blood collection and stem cell harvesting, bone marrow aspirate collection and analysis, culture of non-immortalized cells, preservation of platelet viability, and preservation of DNA integrity.

Preferably, the stabilizing agent comprises or consists of at least one caspase inhibitor, for example a caspase inhibitor which inhibits any or all cysteinyl aspartic acid proteases (Caspases 1-14). Caspase inhibitors are known in the art, as shown for example in U.S. Pat. Nos. 6,153,591, 6,184,210, 6,045,990, 6,355,618, 6,197,750, 5,869,519, 6,200,969, and 6,242,422, and U.S. patent application 2002/0045623, the disclosures of which are hereby incorporated by reference. The stabilizing agent may be in any suitable form including, but not limited to, solution, suspension or other liquid, pellet, tablet, spray-dried, freeze-dried, powder, particle, gel, crystals or lyophilized form. The caspase inhibitor may be in combination with other additives, as discussed below. Because the half-life of many inhibitors may be short, the stabilizing agent is preferably introduced into the collection device in such a form so as to optimize the shelf life of the inhibitor. Lyophilization appears to be particularly useful in that it provides good stability and also allows subsequent sterilization, both of which are key from a standpoint of automation and standardization.

The stabilizing agent may be located on any surface of the collection device. The stabilizing agent may also be located on stoppers and seals for closing such devices or on mechanical, or other, inserts placed within such devices. Preferably, the caspase inhibitor or admixture thereof is located anywhere along at least one interior wall of the collection device or anywhere within the reservoir portion. In addition, some inhibitors may exhibit light sensitivity. Thus, it may be desirable to protect the agent from light. For such inhibitors, use of an opaque tube, e.g., an amber-colored tube, would be advantageous. Alternatively, placing the agent into a capsule that protects it from light exposure, e.g., in powdered form, and then placing the capsule into the tube would also address this issue. Capsulating the agent may also prevent other undesirable interactions between the agent and other elements in the container. Capsule materials that dissolve upon sample collection are well known in the art.

The caspase inhibitor or admixture thereof may be applied to the collection device by any number of methods. For example, the caspase inhibitor or admixture thereof may be spray dried, loosely dispensed or lyophilized over the surface of the interior wall of the collection device. Alternatively, the stabilizing agent, such as when in gel or liquid form, for example, may be positioned in the reservoir portion of the collection or culture device.

The quantity and location of the caspase inhibitor or admixture thereof are determined by several variables, including the mode of application, the specific caspase inhibitor or admixture thereof used, the internal volume and internal pressure of the collection device, and the volume and type of the biological sample drawn or otherwise introduced into the container.

The concentration of the caspase inhibitor or admixture thereof is sufficient to inhibit the caspases and to prevent sample degradation as compared to an identical device without such inhibitor. In addition to the stabilizing agent, the device of the present invention may also contain carrier media (e.g., water, alcohol, dimethylsulfoxide), stabilizing media (e.g., polyvinylpyrollidone, trehalose, mannitol, dextrose) or one or more other additives such for treating the biological sample. Suitable additives include, but are not limited to, alcohols, aldehydes, ketones, organic acids, salts of organic acids, alkali metal salts of halides, organic chelating agents, fluorescent dyes, antibodies, binding agents, anticoagulants such as sodium citrate, heparin, potassium or sodium salts of EDTA and the like, density gradient media and any other reagent or combination of reagents normally used to treat biological samples for analysis. Preferably, the carrier and additives do not inhibit cell function or decrease cell viability. Where the caspase inhibitor or admixture thereof is in tablet form, pharmaceutical tablet disintegrating materials may be included, if desired.

The method of the present invention is performed by obtaining a biological sample and introducing the sample into the container containing the caspase inhibitor. In preferred embodiments, the biological sample is withdrawn from the patient directly into the collection container without any intervening process steps. It has been found that collecting the biological sample directly from the patient, such as when collecting a whole blood sample, and introducing the sample directly into the container containing the stabilizing agent substantially prevents apoptosis that otherwise occurs when the sample is stored before combining it with the caspase inhibitor. The method of the present invention is useful both with open collection systems and with closed collection systems wherein the opening is closed by a closure means.

In one embodiment, the collection device of the present invention is for drawing a whole blood sample directly from a patient for inhibiting apoptosis immediately at the point of collection. The device may be an evacuated, a partially-evacuated or a non-evacuated system for collecting blood. A suitable example of an evacuated system is a closed tube. A manual syringe draw is a suitable example of both a partially-evacuated and a non-evacuated system. Non-evacuated systems can also include automatic draw systems. Evacuated systems are particularly preferred.

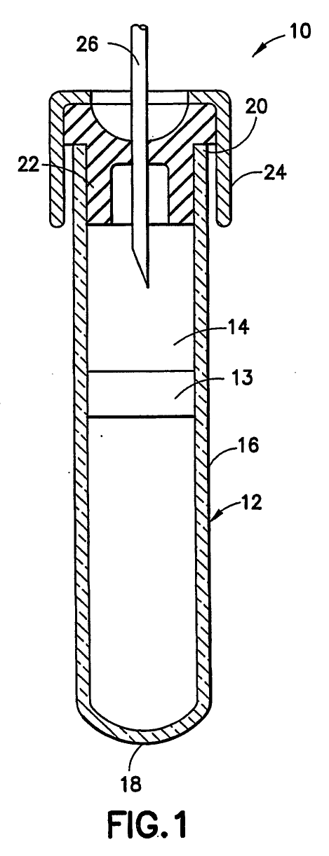

Referring to the drawings in which like reference characters refer to like parts throughout the several views thereof, FIG. 1 shows a typical blood collection device 10, which includes a container 12 defining a chamber 14. In the embodiment illustrated, container 12 is a hollow tube having a side wall 16, a closed bottom end 18 and an open top end 20. Optionally, a separating member 13 is provided within the container chamber 14. Separating member 13 serves to assist in separating components of the sample, for example, by centrifugation. Container 12 is dimensioned for collecting a suitable volume of biological fluid, preferably blood. A closure means 22 for covering open end 20 to close container 12 is necessary where a sterile product is demanded. For conventional tubes, a screw cap is normally sufficient. For evacuated collection tubes, a tight-fitting, elastomeric plug is generally employed to contain the vacuum during the required storage periods. Preferably, closure 22 forms a seal capable of effectively closing container 12 and retaining a biological sample in chamber 14. Closure 22 may be one of a variety of forms including, but not limited to, rubber closures, metallic seals, metal-banded rubber seals and seals of different polymers and designs. A protective shield 24 may overlie closure 22. Container 12 also contains a stabilizing agent in accordance with the present invention.

Container 12 can be made of glass, plastic or other suitable materials. Preferably, container 12 is transparent. Non-limiting examples of suitable transparent thermoplastic materials for container 12 are polycarbonates, polyethylene, polypropylene and polyethyleneterephthalate. Plastic materials can be oxygen impermeable materials or contain an oxygen impermeable or semi-permeable layer. Alternatively, container 12 can be made of a water and air permeable plastic material. The caspase inhibitor or admixture thereof may be provided to the container using any appropriate means. In one aspect, the caspase inhibitor is in a liquid solution and is placed into the container. Subsequently, the solution may be lyophilized by methods that are known in the art, such as, for example, freeze drying. For example, by freezing the solution and then slowly warming after freezing, while simultaneously applying a vacuum, a freeze-dried powder remains in the collection tube. An additive such as an excipient, for example, PVP or trehalose, may also be added to the caspase inhibitor solution prior to freeze drying so that the resulting stabilizing agent is pelletized in the container. Vacuum drying may also be used after adding the stabilizing solution. In another aspect, the caspase inhibitor or admixture thereof is formed into a liquid or solid aerosol and sprayed onto one or more surfaces of the interior of the container.

The pressure in chamber 14 is selected to draw a predetermined volume of biological sample into chamber 14. Preferably, closure 22 is made of a resilient material that is capable of maintaining the internal pressure differential between atmospheric pressure and a pressure less than atmospheric. Closure 22 is such that it can be pierced by a needle 26 or other cannula to introduce a biological sample into container 12 as known in the art. Preferably, closure 22 is resealable. Suitable materials for closure 22 include, for example, silicone rubber, natural rubber, styrene butadiene rubber, ethylene-propylene copolymers and polychloroprene.

Suitable examples of container 12 include single-wall and multi-layer tubes. A more specific example of a suitable container 12 is disclosed in U.S. Pat. No. 5,860,937 to Cohen, which is hereby incorporated by reference in its entirety.

As noted, container 12 may also contain a gel mechanical or other separating member (e.g., filter paper or the like). In such cases, the stabilizing agent may be spray dried and/or lyophilized on an exterior surface of the separation media. Container 12 may also be a collection device for blood plasma preparation. Such a collection device comprises, in addition to the stabilizing agent, an element for separating plasma from human or animal whole blood. The element for separating plasma from whole blood may be a separating member such as a gel formulation or a mechanical media. The gel is desirably a thixotropic polymeric gel formulation. The gel may be a homopolymer or a copolymer and may include silicone-based gels such as, for example, polysiloxanes, or organic hydrocarbon-based gels such as, for example, polyacrylics, polyesters, polyolefins, oxidized cis polybutadienes, polybutenes, blends of epoxidized soybean oil and chlorinated hydrocarbons, copolymers of diacids and propandiols, hydrogenated cyclopentadienes and copolymers of alpha-olefins with dialkylmaleates. The gel desirably isolates the plasma from the cells of the blood sample in the tube by serving as a density separation medium. An example of a suitable plasma preparation tube is disclosed in U.S. Pat. No. 5,906,744 to Carroll et al., which is hereby incorporated by reference in its entirety. In this way, stabilization can be provided both before, during and after centrifugation to separate the plasma from the blood. In the case of a gel separating material, it may be desirable to provide physical/chemical separation between the stabilizing agent and the gel, e.g., use of a capsule as discussed above. For example, if portions of the agent are incorporated into or react with the gel, the effectiveness of the agent may be reduced. For the same reasons, where a mechanical separating element is used, the element is desirable substantially inert to the stabilizing agent, and this reflects a significant advantage of such a separator. Providing a separating element in plasma tubes, versus centrifuging without a separating element, is particularly advantageous. Specifically, because cell lysing may release proteases that induce apoptosis, the better the separation between the cells (i.e., the clotted blood) and the plasma, the better the stability of proteins in the plasma sample. Useful mechanical separators are found, for example, in U.S. Pat. Nos. 6,516,953; 6,406,671; 6,409,528; and 6,497,325, the contents of which are hereby incorporated by reference in their entirety.

Container 12 may also be collection tube for centerifugally separating lymphocytes and monocytes from heavier phases of a sample of whole blood comprising, in addition to the stabilizing agent, a liquid density gradient medium and a means for preventing mixing of the liquid density gradient medium with a blood sample prior to centrifugation. An example of a suitable lymphocyte/monocyte collection tube is disclosed in U.S. Pat. No. 5,053,134 to Luderer et al., which is hereby incorporated by reference in its entirety.

Other commercially available blood collection tubes suitable for use in the present invention include the following, all of which are sold by Becton Dickinson and Company, Franklin Lakes, N.J., with all registrations and trademarks belonging to Becton Dickinson and Company: VACUTAINER® hematology tubes, catalog nos. 367650-1, 367661, 6405, 6385, 6564, 367653, 367665, 367658, 367669, 6450-8, 6535-37 and 367662; VACUTAINER® K₂EDTA tubes, catalog nos. 367841-2, 367856 and 367861; VACUTAINER® PST tubes, catalog nos. 367793-4, 6698, 6595 and 6672; VACUTAINER® CPT tubes, catalog nos. 362753 and 362760-1; VACUTAINER® SST tubes, catalog nos. 367782-89, 6509-17 and 6590-92; and VACUTAINER® ACD tubes catalog nos. 367756, 364012 and 4816.

In another embodiment, the invention provides a kit having at least two containers comprising one or more stabilizing agents. For example, the kit may comprise a primary collection tube, e.g., a plasma separating tube having a separating element therein, and a secondary tube for testing, e.g., for pouring or otherwise dispensing the collected plasma into. Both would have stabilizing agent(s) therein. Optionally, the kit could include a tube-to-tube transfer device to prevent the need for pouring or other unsafe transfer practices, in which case the secondary tube would be at a reduced pressure to draw in the plasma One using such a kit would collect a sample in the primary tube, centrifuge, transfer the sample of interest to the secondary testing tube, and perform the testing. The secondary testing could be of a variety of sizes, depending on the desired testing.

In an embodiment, the container is a tube with two open ends having closures thereon. Such a tube would allow one to sample, e.g., for a plasma separating tube with a separating element therein, either the plasma sample or the clot sample.

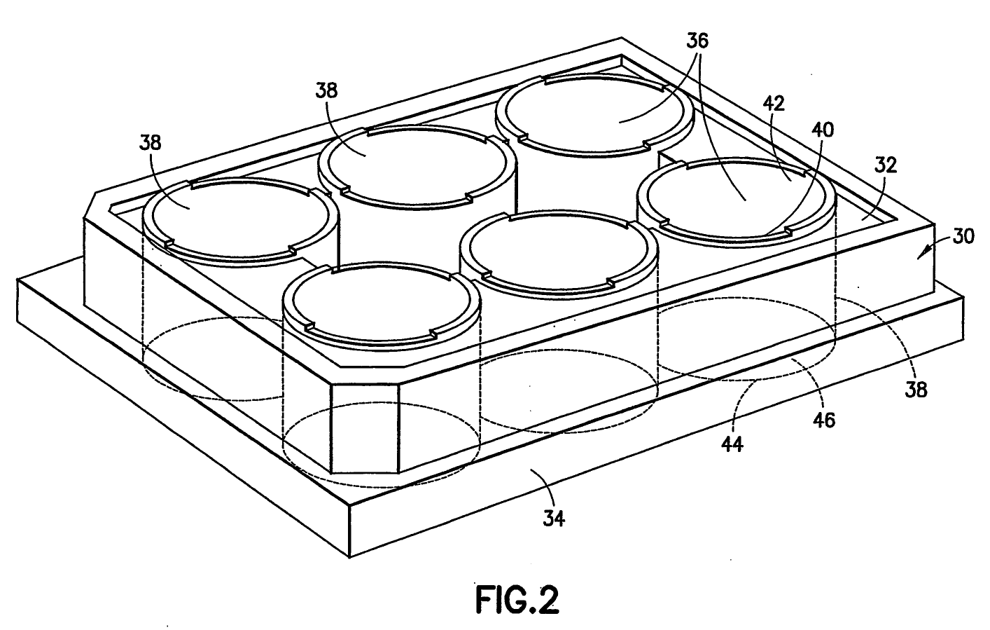

In another embodiment, the collection device of the present invention comprises a tissue culture vessel such as, for example, a single- or multi-well plate, a microtiter plate, a tissue culture plate or flask or the like. A typical test plate generally comprises one or more wells, which are preferably cylindrical. As shown in FIG. 2, a test plate 30 includes an upper surface 32 and a lower surface 34. Test plate 30 further includes a number of wells 36 each comprising a sidewall 38 extending from upper surface 32 of the plate to lower surface 34 of the plate. Each well comprises a top portion 40 and a bottom portion 44. Top portion 40 comprises an open end 42, that extends to bottom portion 44 that comprises a closed end 46. Bottom portion 44 may be flat, conical (pointed) or rounded. The capacity of each well 36 typically ranges from several milliliters (ml) to less than about 0.5 ml. Wells 36 may each accommodate therein a stabilizing agent according to the present invention.

The number of wells 36 in test plate 30 is not critical. There may be any number of wells, although six, twelve, twenty-four, forty-eight and ninety-six well test plates are commonly known and available. In FIG. 2, a six-well test plate is illustrated, merely for exemplary purposes, and the invention is not dependent upon the number of wells. Most standard multi-well plates have the wells arranged in orthogonal rows and columns so as to be able to clearly identify the individual wells being used. Of course, the arrangement of the wells in test plate 30 is not an essential limitation of the present invention because any arrangement of wells is contemplated by the invention.

Plate 30 may be formed from thermoplastic materials by vacuum forming, sheet molding, injection molding or other similar techniques. Suitable thermoplastic materials include, but are not limited to, polystyrene, polyvinylchloride, polycarbonate, polyethyleneterephthalate and the like. Preferably, plate 30 is transparent.

Surrounding the wells and forming the outside border of test plate 30 are sidewalls 38. In the present embodiment, test plate 30 has six (6) sidewalls. Well known test plates are rectangle or quadrilaterally shaped, although for purposes of the present invention the plate may be fabricated in any practical configuration. Examples of suitable test plates containing a plurality of wells are disclosed in U.S. Pat. No. 5,882,922 to Tyndorf et al., U.S. Pat. No. 5,801,055 to Henderson and U.S. Pat. No. 5,681,743 to Brian et al., each of which is hereby incorporated by reference in its entirety.

In yet another embodiment, the collection device according to the present invention may be a sample collection assembly for the collection, transport and dispensing of biological samples. The collection assembly generally includes a plurality of sample wells for collecting individual biological samples. The sample wells are supported in a sample tray in a spaced-apart orientation. The sample tray may be supported within a case that encloses the sample tray and allows the safe and efficient transport of the sample wells. The sample tray is movably accommodated within the case for movement between a first position enclosing the plurality of sample wells, to a second position rendering exteriorly accessible one of the sample wells so that the sample can be manually dispensed from the tray.

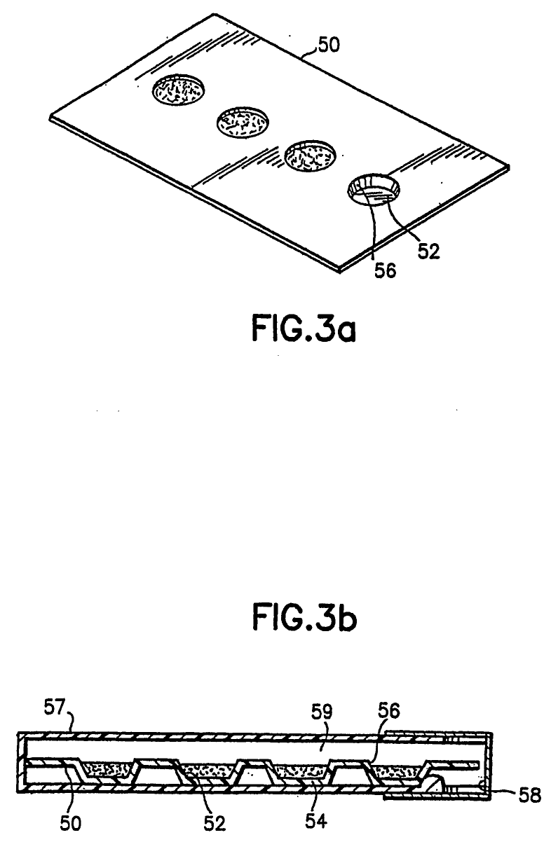

As shown in FIGS. 3a and 3b, sample tray 50 includes a plurality of longitudinally spaced depressions forming specimen collection wells 52. Sample tray 50 may be formed of a suitably deformable plastic material. Wells 52 have a bottom 54 and an open end 56. It is contemplated that the sample wells may be in the shape of open ended cup-like members. Wells 52 are constructed to have sufficient depth so as to retain a suitable volume of a biological sample. Wells 52 may each accommodate therein a stabilizing agent according to the present invention. While tray 50 of the present invention is shown having a single row of wells 52 formed therein, the present invention contemplates that the wells may be provided in any number or any array desirable for a particular testing situation. The sample collection assembly may include a sample collection case 57. Upon collection of a biological sample within wells 52, sample tray 50 may be inserted into the open end 58 of sample collection case 57 and then within the interior 59 of sample collection case 57 until all of wells 52 are enclosed therein. A suitable sample collection assembly is disclosed in U.S. Pat. No. 6,357,583 B1 to Rainen, which is hereby incorporated by reference in its entirety.

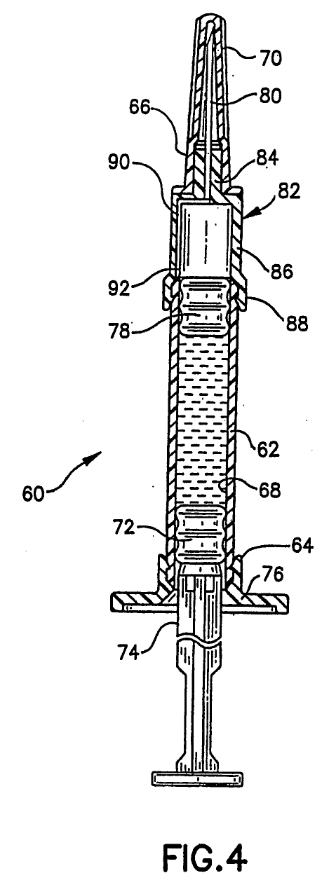

According to another embodiment of the present invention, the collection device comprises a syringe and, more preferably, a pre-filled syringe. A typical syringe comprises a generally cylindrical barrel having opposed proximal and distal ends with at least one chamber formed between the ends for receiving a substance such as a biological sample. A plunger is typically sealably disposed within the barrel and movable with respect thereto, and sealing means may be sealably disposed approximate to the distal end of the barrel. Referring now to FIG. 4, there is shown a syringe 60, which includes an elongate barrel or cylinder 62 having an open, proximal end 64 and a distal end 66, with at least one hollow chamber 68 formed between the proximal and distal ends for receiving a biological sample. In the embodiment illustrated, distal end 66 includes a needle guard 70. The needle guard keeps the syringe, as well as the needle, sterile during storage.

The barrel of the syringe includes a stabilizing agent. Preferably, the barrel of the syringe is pre-filled with the stabilizing agent. Pre-filled syringes, as the term is known in the art, are syringes that are filled by the manufacturer and shipped to the health care provider ready for use.

A plunger 72 may be situated at open, proximal end 64. Plunger 72 can be moved by means of a plunger rod 74, which is secured to the plunger, for example, by screwing. At the same end where the plunger is situated, the barrel may have a fingergrip 76, which is secured to the barrel according to the so-called snap-cap principle. Fingergrip 76 preferably consists of slightly resilient material, for example plastics. In another embodiment (not shown), the fingergrip is a flange-like part of the barrel projecting radially outwards. Of course, other constructions known to those skilled in the art are possible.

A stopper 78, which closes the barrel, may be situated in the end of the barrel remote from the plunger. The plunger and the stopper are preferably manufactured from an elastic material and, most preferably, from rubber of a pharmaceutical quality.

In the embodiment illustrated, an injection needle 80 is secured to the barrel by means of a needle holder 82. The needle holder has a neck 84, which holds the needle, a shaft 86 and a collar 88. The needle holder is preferably manufactured from slightly resilient material that has resistance to deformation such as, for example, plastics, and is secured to the end of the barrel by means of a snap-cap construction. In the alternative, the needle holder may be secured to the barrel by means of a screwed or adhesive connection or, when the barrel also comprises a collar, by means of a clamping ring; in the latter embodiment, the needle holder may also be flanged around a collar of the barrel.

Although the syringe barrel illustrated in this embodiment includes a locking Luer-type collar 88, it is within the purview of the present invention to include syringe barrels without a collar, syringe barrels having an eccentrically positioned nozzle, and various other nozzle-like structures adapted to accept, either permanently or removably, a needle cannula or needle cannula assembly. It is only required that there is an aperture on the distal end of the syringe barrel in fluid communication with the interior of the syringe barrel.

One or more slots 90 may be recessed in the inner wall of shaft 86 and the rear face of neck 84. The slot or slots extend into the rear end of the cannula. In cross-section, the slots may be parts of a circle, but other shapes are also possible, provided the size is such that sufficient injection liquid can be readily passed through; this is achieved if the diameter of the slot or the overall cross-section of the slots is at least as large as that of the cannula. Shaft 86 of needle holder 82 is constructed so that when stopper 78 slides axially forward, it is received, with friction, by the shaft; therefore, apart from slots 90 recessed in the shaft, the inside diameter of the shaft is approximately as large as that of barrel 62. Shaft 86 of needle holder 82 is slightly longer than stopper 78 so that the part 92 of the slot(s) adjoining the barrel is free when the stopper is moved forward against the rear wall of the neck of the needle holder. If desired, needle guard 70 may be constructed to also serve as a plunger rod. In that case, prior to use of the syringe, the needle guard is removed from the needle and secured at the other end of the syringe to the plunger.

Generally, a syringe comprising a needle protector has a safety member, which indicates whether the needle protector has previously been removed. Such a safety member in the form of a cap is described, for example, U.S. Pat. No. 3,995,630.

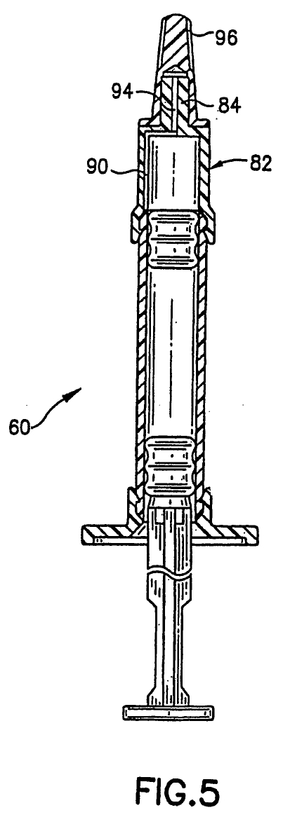

In further embodiments, the syringe is not stored with a needle in position, i.e., it is a needleless syringe as known in the art. This is illustrated in FIG. 5. With such a syringe, before use, the needle is positioned on neck 84 of needle holder 82 by means of a needle hub. A so-called Luer cone is preferably used for this connection. In this embodiment, aperture 94 in the neck of the needle holder is closed on the outside by a protective cap 96, which ensures the sterility of the syringe as well as the needle holder. Slot 90 recessed in the needle holder projects into the end of the neck aperture.

An example of a suitable syringe is disclosed in U.S. Pat. No. 6,027,481 to Barrelle et al., which is hereby incorporated by reference in its entirety. Other examples of suitable syringes are disclosed in, for example, U.S. Pat. No. 4,964,866 to Szwarc, U.S. Pat. No. 4,986,818 to Imbert et al., U.S. Pat. No. 5,607,400 to Thibault et al. and U.S. Pat. No. 6,263,641 B1 to Odell et al., each of which is hereby incorporated by reference in its entirety.

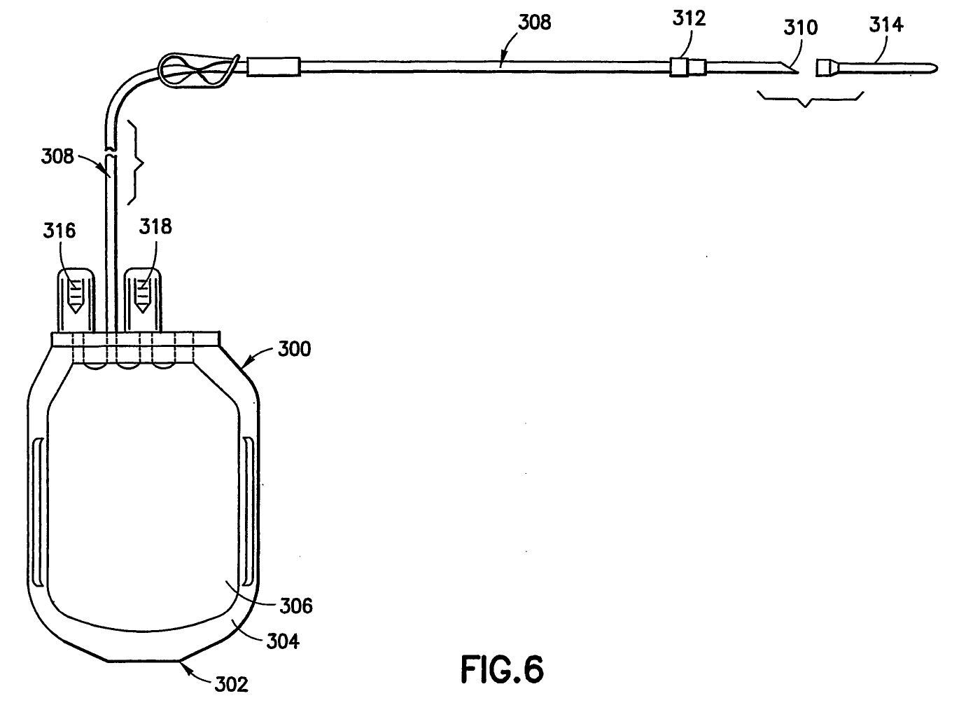

The collection device of the present invention may also comprise a collection bag suitable for holding a biological sample such as, for example, a blood collecting bag, a blood plasma bag, a buffy coat bag, a platelet bag or the like. For ease of description, a blood collecting bag will now be described with reference to FIG. 6.

FIG. 6 illustrates a blood collecting bag 300 for accommodating collected blood. Blood collecting bag 300 has a body 302 formed by superposing a pair of identically cut pieces of a sheet material made of a resin, which will be more specifically described hereinafter, and possessed of flexibility and fusing (i.e., heat fusion, high frequency fusion or the like) or adhesively joining to each other the periphery of the sealing portion 304 of each of the pieces of sheet material. A blood-accommodating portion 306 accommodating collected blood is formed at an inner portion surrounded with sealing portion 304 of body 302. Blood collecting bag 300 preferably contains a stabilizing agent in accordance with the present invention.

One end of the flexible tube 308 communicating with blood-accommodating portion 306 is connected with body 302 at an upper portion thereof. A blood collecting needle 310 is installed at the other end of flexible tube 308 through a hub 312. A cap 314, which is to cover blood collecting needle 310, may be installed on hub 312. Two openings 316 and 318, each sealed with a peel tab, may be formed at an upper portion of body 302 such that they can be opened.

The composition, characteristics and the like of the material of the sheets composing body 302 of blood collecting bag 300 are not limited to specified ones. In this case, as the sheet material composing blood collecting bag 300, soft polyvinyl chloride or materials containing the soft polyvinyl chloride as their main component is preferably used. For example, a copolymer containing the soft polyvinyl chloride as its main component and a small amount of macromolecular material, a polymer blend, a polymer alloy and the like can be used. As the plasticizer for the soft polyvinyl chloride, dioctylphthalate (DEBHP, di(2-ethylhexyl)phthaldte) and (DnDP, di(n-decyl)phthalate) can be preferably used. The content of such a plasticizer in the polyvinyl chloride is preferable to be in the approximate range of 30 to 70 parts by weight, based on 100 parts by weight of polyvinyl chloride.

The other substances which are effectively usable for the sheet material of blood collection bag 300 are polyolefins, ie., the products of homopolymerization or copolymerization of such olefins or diolefins as ethylene, propylene, butadiene, and isoprene. As typical examples, polyethylene, polypropylene, ethylene vinyl acetate copolymer (EVA), polymer blends formed between EVA and various thermoplastic elastomers, and arbitrary combinations thereof may be cited. Besides, such polyesters as polyethylene terephthalate (PET), polybutylene terephthalate (PBT), poly-1,4-cyclohexane dimethyl terephthalate (PCHT) and polyvinylidene chloride are also usable.

In yet another embodiment, the collection device of the present invention may be a laboratory vessel that contains caspase inhibitor or admixture thereof. Particular vessels that can be used in accordance with the present invention include, for example, vials, flasks, spinner flasks, roller bottles, microscope slides, microscope slide assemblies, sample chambers for analytical devices, tapes, laminates, arrays, catheters, pipettes, tubing and the like. Laboratory vessels according to the present invention have at least one operational surface. Many vessels according to the invention have at least one interior wall, which defines a reservoir portion for containing the biological sample, and at least one opening in communication with the reservoir portion.

Plastic or glass is often used to manufacture the laboratory vessels. Some preferred materials used to manufacture laboratory vessels include polypropylene, polyethylene, polyethyleneterephthalate, polystyrene, polycarbonate and cellulosics. Because polypropylene is inexpensive, it is a particularly preferred material for laboratory vessels used for handling and transporting minute and precise amounts of biological sample.

Examples of other suitable materials for the laboratory vessels of the present invention include polyolefins, polyamides, polyesters, silicones, polyurethanes, epoxies, acrylics, polyacrylates, polyesters, polysulfones, polymethacrylates, PEEK, polyimide and fluoropolymers. Glass products including silica glass are also used to manufacture laboratory vessels.

In another embodiment, cells stabilized by the articles and/or processes of the inventions are used as therapies. For example, cells from umbilical cord blood have been used as therapy, e.g., by transplanting sub cells to patients having genetic or blood disorders. Stem cells found in the cord bloods are believed to replace or supplement non-functioning or malfunctioning cells of the recipient. It is also believed that such stem cells may help regenerate damaged tissues.

According to the invention, umbilical cord blood (or placental blood) is stabilized upon collection by use of a container comprising one or more caspase inhibitors, as discussed above. The blood is then transplanted to a human being, either after being cryopreserved, or fresh. (Cryopreservation techniques are know to those skilled in the art.)

Similary, it is possible to collect a product of leukapheresis in a container comprising one or more caspase inhibitors, and administering that product to a patient, again either after being cryopreserved or fresh. (Leukapheresis, as know in the art, is a process in which blood is drawn, a specific cell product is separated out, and the remainder of the blood is returned to the subject.

By using a collection container comprising one or caspase inhibitor, the stability of stem cells would be expected to improve, relative to an identical container without such caspase inhibitors. As a result, the therapeutic effect of the stabilized product would be expected to improve, as well. Advantagously, the caspase inhibitors do not detrimentally effect cryopreservation steps, and are substantially benign to the human body upon injection.

What is claimed is:

1. An apparatus for containing a biological sample, comprising:
a container having a reservoir portion for receiving the sample; and
a stabilizing agent in the reservoir of the container, the agent comprising a caspase inhibitor.

2. The apparatus of claim 1, wherein the container is selected from the group consisting of tubes, closed system blood collection devices, collection bags, syringes, pre-filled syringes, catheters, microtiter plates, multi-well collection devices, flasks, spinner flasks, roller bottles, vials, pipettes, pipette tips and tissue and other biological sample collection containers.

3. The apparatus of claim 1, wherein the container is a tube having a first end and a second end.

4. The apparatus of claim 3, further comprising a separating member disposed in the container.

5. The apparatus of claim 4, wherein the separating member is a mechanical separating element.

6. The apparatus of claim 5, wherein the mechanical separating element is at least partially coated with the at least one stabilizing agent.

7. The apparatus of claim 5, wherein the mechanical separating element is substantially inert with respect to the stabilizing agent.

8. The apparatus of claim 4, wherein the separating member is a gel.

9. The apparatus of claim 8, wherein the gel separating member is physically separated from the stabilizing agent.

10. The apparatus of claim 1, wherein the stabilizing agent is in a form selected from the group consisting of a solution, suspension or other liquid, a pellet, a tablet, a capsule, a spray-dried material, a freeze-dried material, a powder, a particle, a gel, crystals or a lyophilized material.

11. The apparatus of claim 10, wherein the stabilizing agent is lyophilized.

12. The apparatus of claim 1, wherein the caspase inhibitor inhibits one or more cysteinyl aspartic acid proteases.

13. The apparatus of claim 1, wherein the stabilizing agent comprises more than two caspase inhibitors.

14. The apparatus of claim 1, further comprising a carrier media.

15. The apparatus of claim 1, further comprising a stabilizing media.

16. The apparatus of claim 15 wherein the stabilizing media is trehalose.

17. The apparatus of claim 1, further comprising at least one antioxidant.

18. The apparatus of claim 1, further comprising at least one reducing agent.

19. The apparatus of claim 1, further comprising at least one buffering agent.

20. The apparatus of claim 3, further comprising a closure means for sealing the first end.

21. The apparatus of claim 20, wherein the tube is partially evacuated.

22. The apparatus of claim 21, wherein the stabilizing agent is lyophilized.

23. The apparatus of claim 22, wherein the stabilizing agent comprises more than two caspase inhibitors.

24. The apparatus of claim 23, wherein the tube further comprises an anticoagulant.

25. The apparatus of claim 24, wherein the anticoagulant is spray-dried onto at least a portion of an interior wall.

26. The apparatus of claim 25, wherein the anticoagulant comprises a salt of EDTA.

27. The apparatus of claim 24, wherein the anticoagulant comprises heparin.

28. A tube for collecting and stabilizing a biological sample, comprising:
a first end, a second end and at least one interior wall defining a reservoir portion for receiving the sample;
at least one stabilizing agent in the reservoir of the container, the stabilizing agent comprising a caspase inhibitor;
a thixotropic polymeric gel in the reservoir; and
an element for maintaining separation of the stabilizing agent and the gel.

29. The tube of claim 28, wherein the element for maintaining separation is a capsule.

30. The tube of claim 29, further comprising a closure means for sealing the first end.

31. The tube of claim 30, wherein the closure means is pierceable by a needle for supplying the sample to the tube.

32. The tube of claim 30, wherein the tube is partially evacuated.

33. The tube of claim 32, wherein the stabilizing agent is lyophilized.

34. The tube of claim 33, wherein the stabilizing agent comprises more than two caspase inhibitors.

35. The tube of claim 33, wherein the tube further comprises an anticoagulant spray-dried onto at least a portion of the interior wall.

36. A tube for collecting and stabilizing a biological sample, comprising:
a first end, a second end and at least one interior wall defining a reservoir portion for receiving the sample;
at least one stabilizing agent in the reservoir of the tube the agent comprising a caspase inhibitor; and
a mechanical separating element in the reservoir.

37. The tube of claim 36, wherein the mechanical separating element is substantially inert with respect to the stabilizing agent.

38. The tube of claim 36, further comprising a closure means for sealing the first end.

39. The tube of claim 38, wherein the closure means is pierceable by a needle for supplying the sample to the tube.

40. The tube of claim 38, wherein the tube is partially evacuated.

41. The tube of claim 40, wherein the stabilizing agent is lyophilized.

42. The tube of claim 41, wherein the stabilizing agent comprises more than two caspase inhibitors.

43. The tube of claim 41, wherein the tube further comprises an anticoagulant spray-dried onto at least a portion of the interior wall.

44. A kit for collecting and storing a biological sample for subsequent testing, comprising:
a primary collection tube having a separator element therein; and
a secondary tube;
wherein the primary collection tube and the secondary tube contain one or more stabilizing agents, the agents comprising one or more caspase inhibitors.

45. The kit of claim 44, wherein the separator element is a mechanical separating element.

46. The kit of claim 45, wherein the mechanical separating element is at least partially coated with the one or more stabilizing agents.

47. The kit of claim 46, wherein the mechanical separating element is substantially inert with respect to the one or more stabilizing agents.

48. The kit of claim 44, wherein the separator element is a gel, and the gel is physically separated from the stabilizing agent.

49. The kit of claim 44, further comprising a tube-to-tube transfer device.

50. The kit of claim 49, wherein the second tube is maintained at a pressure to draw the sample from the first tube through the tube-to-tube transfer device and into the second tube.

51. A method of stabilizing a biological sample, comprising:
providing a sample collection container; and
disposing the biological sample into the collection container such that the sample is contacted with a stabilizing agent comprising a caspase inhibitor.

52. The method of claim 51, wherein the sample collection container includes the stabilizing agent before collecting the biological sample.

53. The method of claim 51, wherein the disposing of the biological sample into the container and the contacting of the sample with the stabilizing agent are performed in the same collection container.

54. The method of claim 53, wherein the collection container is evacuated and has a predetermined internal pressure sufficient to draw a predetermined volume of the sample into the collection container.

55. The method of claim 51, wherein the collection container is selected from the group consisting of tubes, closed system blood collection devices, collection bags, syringes, microtiter plates, multi-well collection devices, flasks, spinner flasks, roller bottles and vials.

56. The method of claim 51, wherein the stabilizing agent comprises more than two caspase inhibitors.

57. The method of claim 51, wherein the biological sample is selected from the group consisting of whole blood or a component thereof, umbilical cord or placental blood, red blood cell concentrates, platelet concentrates, leukocyte concentrates, plasma, serum, urine, bone marrow apirates, cerebral spinal fluid, tissue, cells, feces, saliva and oral secretions, nasal secretions and lymphatic fluid.

58. The method of claim 57, wherein the biological sample is whole blood.

59. The method of claim 58, wherein the whole blood is collected from a patient directly into the collection container.

60. The method of claim 59, wherein the collection container includes the stabilizing agent before the blood is collected from the patient.

61. A method for making a collection container for collecting a biological sample, comprising:
providing a collection container;
disposing a stabilizing agent comprising at least one caspase inhibitor into the container;
lyophilizing the stabilizing agent;
evacuating and sealing the container; and
sterilizing the container.

62. The method of claim 61, wherein the collection container is a tube.

63. The method of claim 62, further comprising placing into the tube a separating member.

64. The method of claim 63, wherein the separating member is a mechanical separating element.

65. The method of claim 63, wherein the separating member is a gel.

66. A method for treating, comprising the steps of:
collecting a cell population that comprises hematopoetic stem cells, wherein the cell population is collected in a container comprising one or more caspase inhibitors; and administering at least a portion of the collected cell population into a patient.

67. The method of claim 66, wherein umbilical cord blood or placental blood is collected into the container.

68. The method of claim 66, wherein at least a portion of the one or more caspase inhibitors are administered into the patient along with the at least a portion of the collected cell population.

69. The method of claim 66, further comprising the step of cryopreserving the collected cell population, and thawing the collected cell population, prior to the step of administering.

70. The method of claim 66, wherein the container comprises at least two caspase inhibitors.

71. The method of claim 69, wherein at least a portion of a cryopreservative used in the cryopreserving step is administered into the patient along with the at least a portion of the one or more caspase inhibitors and the at least a portion of the collected cell population.

72. A method for treating, comprising the steps of:
performing a leukapheresis process to collect a cell population that comprises hematopoetic stem cells, wherein the cell population is collected in a container comprising one or more caspase inhibitors; and
administering at least a portion of the collected cell population into a patient.

73. The method of claim 72, wherein at least a portion of the one or more caspase inhibitors are administered into the patient along with the at least a portion of the collected cell population.

74. The method of claim 72, further comprising the step of cryopreserving the collected cell population, and thawing the collected cell population, prior to the step of administering.

75. The method of claim 72, wherein the container comprises at least two caspase inhibitors.

76. The method of claim 74, wherein at least a portion of a cryopreservative using in the cryopreserving step in administered into the patient along with the at least a portion of the one or more caspase inhibitors and the at least a portion of the collected cell population.
